(19) United States
(12) Patent Application Publication
(10) Pub. No.: US 2019/0000681 A1
(43) Pub. Date: Jan. 3, 2019

(54) VAGICREAM VAGINAL CREAM

(71) Applicant: Michele Soughayar Lovink, Washington, DC (US)

(72) Inventor: Michele Soughayar Lovink, Washington, DC (US)

(21) Appl. No.: 15/640,864

(22) Filed: Jul. 3, 2017

(51) Int. Cl. A61F 13/20; A61K 31/566; A61K 9/00

(52) U.S. Cl. CPC A61F 13/2074; A61K 9/0034; A61K 31/566

(57) ABSTRACT

THIS INVENTION IS A NOVEL PRACTICAL WAY OF ADMINISTERING PHARMACEUTICAL FORMULATION COMPRISING CONJUGATED ESTROGEN CREAM IN VARIOUS DOSAGES, TO BE GIVEN VAGINALLY THROUGH INSERTION OF A DISPOSABLE TAMPON FOR THE TREATMENT OF VULVOVAGINAL ATROPHY, VAGINAL DRYNESS AND SEXUAL DYSFUNCTION IN POSTMENOPAUSAL WOMEN.

### CROSS REFERENCE TO RELATED APPLICATIONS

THIS APPLICATION IS A NON PROVISIONAL APPLICATION AND CLAIMS PRIORITY TO THE FOLLOWING: U.S. PROVISIONAL PATENT APPLICATION No. 62/493,767 FILED Jul. 15, 2016, ENTITLED DELIVERED IN A DISPOSABLE PLASTIC TAMPON, IN ORDER TO EASE VAGINAL DISCOMFORT DURING INTERCOURSE. THIS COMBINATION PRODUCT HAS BEEN PRESENTED TO THE FDA FOR APPROVAL ON May 13, 2016. (FDA CLASSIFICATION EMAIL INCLUDED) STATEMENT UNDER 37 CFR 1.55 OR 1.78 FOR AIA (FIRST INVENTOR TO FILE)

### BACKGROUND

### Field

POSTMENOPAUSAL WOMEN OFTEN EXPERIENCE VAGINAL DYSFUNCTIONS, DUE TO DECREASED LEVEL OF VARIOUS HORMONES SUCH AS ESTROGENS; IN PARTICULAR VAGINAL DRYNESS AND DYSPAREUNIA, CAUSING PAIN DURING INTERCOURSE AND LEADING TO LOWER SEXUAL DESIRE AND PERFORMANCE.

THESE SYMPTOMS OFTEN LEAD TO POOR BODY IMAGE, LACK OF LIBIDO, RELATIONSHIP ISSUES AND MENTAL HEALTH ISSUES.

VAGINAL ATROPHY OFTEN INCREASES THE LIKELIHOOD OF URINARY TRACT INFECTIONS.

THE PRESENT INVENTION DESCRIBES THE EFFECT OF CONJUGATED ESTROGEN ON THE LAYERS OF THE VAGINA AND VULVA, AND THE EASE OF USING THIS INVENTION.

THE METHOD OF DELIVERY OF THIS INVENTION IS TO PROVIDE A LOCAL VAGINAL THERAPY; IT IS EASY, CONVENIENT, HYGIENIC, DISCREET, AND COMFORTABLE TO USE.

IT PROVIDES EXCELLENT PATIENT ACCEPTABILITY.

THE PRESENT AND USUAL METHOD OF USAGE, ARE REUSABLE APPLICATOR. THESE APPLICATORS ARE MESSY AND NEED TO BE WASHED AFTER EVERY USE. THE METHOD OF FILLING THE REUSABLE APPLICATOR IS MESSY AND LEAVES THE CREAM TUBE OOZING CREAM AFTER USAGE.

THIS PROPOSED INVENTION, OFFERS DISPOSABLE PREFILLED APPLICATORS, DISCREET, HYGIENIC AND EASY TO USE; THUS LEADING TOA BETTER PATIENT ACCEPTABILITY AND COMPLIANCE IN USAGE OF THE MEDICAMENT.

### Background of this Invention

VAGINAL DRYNESS AFFECTS APPROXIMATELY 50% OF POSTMENOPAUSAL WOMEN BETWEEN THE AGES OF 50 TO 60 YEARS AND 72% AFTER 70 YEARS (ROSSIN-AMAR, 2000, GYNECOL OBSTET FERTIL, 28(3): 245-249 COMMONLY, AROUND 80% OF THESE WOMEN EXPERIENCE UROGENITAL DISORDER, MAINLY VAGINITIS AND DYSPAREUNIA (PANDIT AND OUTLANDER, 1997, AM J MED SIC, 314 (4); 228-31).

### MEDICAMENT DELIVERY DEVICE AND ITS METHOD

THE PRESENT INVENTION ISA NOVEL AND EASIER WAY OF ADMINISTERING A MEDICAMENT TO THE HUMAN AND ANIMAL BODY TO TREAT OR PREVENT DISEASE.

Benign gynecological conditions are treated primarily via hormonal oral administration, provided in pill, capsule, powder or liquid form. The medicament is then absorbed through the digestive system and passes into the blood stream via the liver “first pass liver metabolism”, thus requiring larger drug dosages. Larger drug dosages might cause side effects.

The delivery of the drug in the present invention is directly into the vagina to treat gynecological disorders; this means the drugs are not subjected to liver metabolism as in the case of drugs taken orally.

In general, creams and gels are viewed by patients to be messy and unhygienic.

The aim of this invention is to ease and minimize these difficulties.

Many improvements and modifications could be made without departing from the proposed invention. For example regarding the size of the disposable applicator or its composition: how large or long it should be, or from what material it should be made of.

The invention claims:

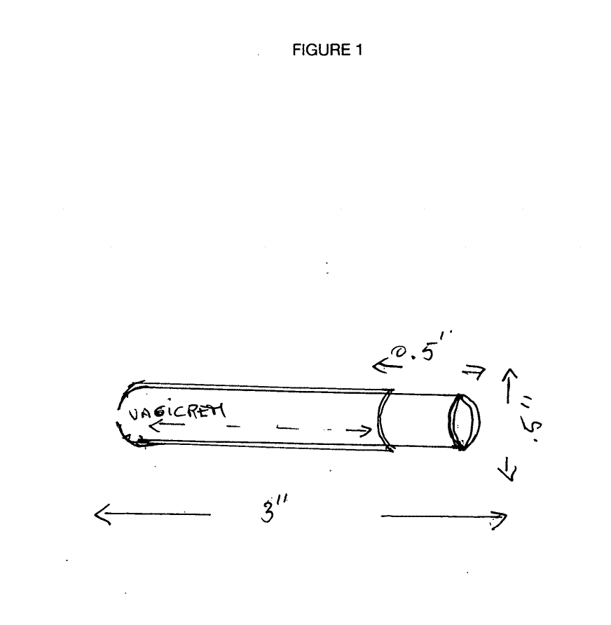

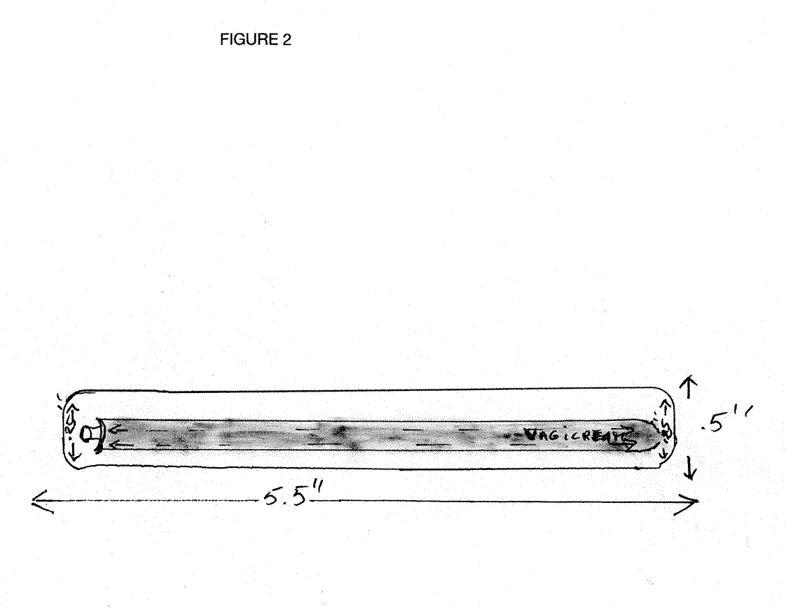

1: A disposable applicator for the delivery of conjugated estrogen cream; the disposable applicator could be in any material, length or width. The delivery device introduces the medicament cream into the vagina and targets the tissues of the vagina and vulva of a female mammal.
A schematic of the disposable applicator is shown in FIGS. 1 and 2.
The disposable device is introduced into the body via the vagina; the cream is injected and the applicator is disposed off.
When the conjugated estrogen cream is inserted into the highly vascularized vagina smooth muscles it causes a good pharmacokinetics effect, and good drug delivery to these tissues. The drug is able to go through the vaginal tissue, the female vulva and vagina without having to go through the first liver metabolism. In particular, this disposable applicator can be used to deliver drugs that cannot be delivered orally.
Alternatively, the disposable delivery device may be inserted into the vagina in any suitable location or other body position.
A non sharp point enables the vagina tissue to be minimally disrupted to allow insertion of the device, the medicament is then squeezed into the vagina, with minimal tissue damage. The head portion of the disposable device is substantially flat, and allows the device to be easily inserted into the smooth muscle of the vagina tissue.
This is advantageous as the location of the disposable device can be discreetly and hygienically used.
The overall disposable device of this invention is advantageous as there will be less discomfort and more compliance by the person using this disposable device and medicament.
This disposable device may be used to deliver conjugated estrogens hormones such as Premarin or other hormonal creams to treat gynecological disorders or vaginitis, vulva atrophy and lack of libido.
This disposable invention could be further used for other ailments such as endometriosis, cardiovascular agents, anti-hypertensive agents, essential amino-acids, fats and vitamins, probiotics, probiotics, acidifiers and chemotherapeutic agents.
Preferably, the medicament and present invention is a therapy for estrogen dependent disorders of the pelvic, causing lack of libido and discomfort during intercourse.
Preferably, the medicament used is in an amount from 5%, 10%, 15%, 20% 25% of the medicament in the device for a period of 1 week, 2 weeks, 1 months, 2 months, 3 months, 4 months, 6 months, 1 year or more to be used 3 times a week.
FIG. 1 is an illustration of the disposable drug delivery device according to the invention for the delivery of the drug to the tissues of the vagina and the pelvic area.
FIG. 2 shows an illustration of the present invention where the device may be shorter in length, approximately 10 cm to 13 cm in length and the head portion would be larger around 0.5 cm to 1.5 cm in width.
This disposable drug delivery device, delivers medicaments to the vaginal cavity, mucosa and pelvic tissue, making it advantageous for treating gynecological disorders and vaginitis.
The drug delivery may be in any suitable pharmaceutically acceptable agent such as, a hydrogel carrying the active agent, a silicone based m material, polysaccharide or carbohydrate material, microspheres, polymeric material or plastics material elastomer, proteinaceous material, polyethylene glycol (PEG) material into the disposable device. This is illustrated in FIG. 2, for the delivery of the drug to the vaginal cavity.
This present invention is an easy method of drug delivery of the active drug into the vaginal cavity, the pelvic region and surrounding soft tissues and blood vessels.
The vaginal cavity has few somatic pain fibers and insertion will cause minimal discomfort to the patient.
This method of delivery is advantageous over oral subcutaneous or reusable devices used previously which are painful non hygienic and often cause patient non compliance.
When inserted into the vaginal cavity the disposable device will not be felt by the patient. The present invention will be acceptable to women of a range of religious faiths.
Delivery of the drug through the vaginal cavity minimizes the risk of poor absorption as the absorption of the active ingredients is facilitated by the high local blood flow. The drug delivery into the vaginal cavity is enabled through the epithelium lining the vagina and the local tissues around.
Drug delivery directly into the vaginal cavity will likely require a lesser amount of a drug in order to achieve a clinical effect, therefore reducing risks of side effects.
The disposable drug delivery device suitable for delivery of drugs to the vaginal cavity in FIG. 1, typically has a diameter of 1 cm and a length of 10 cm. These diameters and lengths are, only for guidance, and other useful dimensions might be useful for this purpose.
The disposable present invention may be used to deliver a wide range of drugs; in particular to deliver drugs which cannot be delivered orally.
Currently, oral daily doses of conjugated estrogens are 0.625 mg. An analogue of 10% of the drug is used for vaginal delivery compared to oral doses.

The invention claims:

1: A disposable applicator for the delivery of conjugated estrogen cream; the disposable applicator could be in any material, length or width. The delivery device introduces the medicament cream into the vagina and targets the tissues of the vagina and vulva of a female mammal.
A schematic of the disposable applicator is shown in FIGS. 1 and 2.
The disposable device is introduced into the body via the vagina; the cream is injected and the applicator is disposed off.
When the conjugated estrogen cream is inserted into the highly vascularized vagina smooth muscles it causes a good pharmacokinetics effect, and good drug delivery to these tissues. The drug is able to go through the vaginal tissue, the female vulva and vagina without having to go through the first liver metabolism. In particular, this disposable applicator can be used to deliver drugs that cannot be delivered orally.
Alternatively, the disposable delivery device may be inserted into the vagina in any suitable location or other body position.
A non sharp point enables the vagina tissue to be minimally disrupted to allow insertion of the device, the medicament is then squeezed into the vagina, with minimal tissue damage. The head portion of the disposable device is substantially flat, and allows the device to be easily inserted into the smooth muscle of the vagina tissue.
This is advantageous as the location of the disposable device can be discreetly and hygienically used.
The overall disposable device of this invention is advantageous as there will be less discomfort and more compliance by the person using this disposable device and medicament.
This disposable device may be used to deliver conjugated estrogens hormones such as Premarin or other hormonal creams to treat gynecological disorders or vaginitis, vulva atrophy and lack of libido.
This disposable invention could be further used for other ailments such as endometriosis, cardiovascular agents, anti-hypertensive agents, essential amino-acids, fats and vitamins, probiotics, probiotics, acidifiers and chemotherapeutic agents.
